(19) United States
(12) Patent Application Publication
(10) Pub. No.: US 2006/0062832 A1
(43) Pub. Date: Mar. 23, 2006

(54) Sanitizing wipes with microbicidal properties cross-reference to related applications

(76) Inventor: John Alex Lopes, Troy, MI (US)

Correspondence Address: Arnold S. Weintraub;The Weintraub Group, P.L.C, Suite 240, 32000 Northwestern Highway, Farmington Hills, MI, 48334, US

(21) Appl. No.: 11/130,455

(22) Filed: May 16, 2005

Related U.S. Application Data
(60) Provisional application No. 60/611,559, filed on Sep. 20, 2004.

(51) Int. Cl. A61K 9/70

(52) U.S. Cl. 424/443

(57) ABSTRACT

A biodegradable sanitizing and microbicidal wipe composition using food additive ingredients or ingredients regarded as safe by the FDA. The wipes hereof are safe for sanitizing and disinfecting food, food contact, and environmental surfaces and skin, hands and other body parts.

### CROSS-REFERENCE TO RELATED APPLICATIONS

This application is a completion application of co-pending U.S. Provisional application Ser. No. 60/611,559, filed Sep. 20, 2004, for “Sanitizing Wipes with Microbicidal Properties”, the entire disclosure of which is hereby incorporated by reference.

### BACKGROUND OF INVENTION

1. Field of Invention

This invention relates to the preparation of sanitizing and microbicidal wipes useful for cleaning, sanitizing and disinfecting environmental, food contact, food and body surfaces including skin, hands and other body parts, in order to prevent, to reduce or to eliminate the risk of infection and other associated complications arising from microbial contamination. More particularly, the present invention relates to sanitizing and microbicidal wipes based on the combined cleaning and microbicidal properties of ingredients considered food additive or Generally Regarded As Safe (GRAS) by the US FDA.

2. Prior Art

Antibacterial and bactericidal wipes used for cleaning environmental, food contact surfaces and personal and health care applications incorporate various antimicrobial agents in addition to anionic and nonionic surface-active agents at pH greater than neutrality. The commercially available sanitizing wipes use ethyl and isopropyl alcohols, hydrogen peroxide, quaternary ammonium compounds (Qacs), benzyl-4-chlorophenol and phenylphenol as active microbicidal agents. The majority of the wipes use Qacs alone or in combination with isopropyl alcohol. Some of these commercially available brands with their active ingredients are as follows: LYSOL® (alkyl dimethyl benzyl ammonium chlorides), CHLOROX® (dimethyl benzyl ammonium chloride), LEVER® 2000 and EQATE® (benzethonium chloride), ALPET® D2 and SANI-CLOTH wipe (isopropyl alcohol and a mixture of Qacs), PROSSPRAY WIPE (bezyl-4-chlorophenol and phenylphenol, and PURELL® (ethyl alcohol 62%). Some other wipes use hydrogen peroxide.

High concentrations of alcohols required for sanitizing activity are flammable. Repeated use of volatile alcohol-based wipes can pollute air in enclosed spaces, such as homes, restaurants, submarines, space vehicles, including the shuttle, the International Space Station and the planetary outposts. Hydrogen peroxide is corrosive and less stable at the concentrations needed for sanitizing purposes. Qacs have high retention on metal, glass and plastic surfaces. Qacs can build up cumulative residue on surfaces after each application. Also, Qacs are less effective against gram-negative bacteria. Some of the Qacs are inhibitors of butyl cholinesterase found in mammalian blood plasma, liver, pancreas, intestinal mucosa and the white matter of the central nervous system. Since acetyl cholinesterase plays a crucial role in neurotransmission, inhibition of butyl cholinesterase may have an adverse effect on physiological functions. Toxic chemicals such as bezyl-4-chlorophenol and phenylphenol are undesirable in sanitizing wipes for use on food, food contact or skin surfaces. Wipes containing iodine-based disinfecting agents are also undesirable because of staining properties or their role in thyroid function.

Cleaning ingredients used in wipes include surface-active agents to reduce surface tension and remove oily soil deposits. Anionic surface-active agents are commonly used in cleaning compositions and at low pH these surface-active agents are also used for the preparation of sanitizers or disinfecting solutions. However the surface-active agents used in commercially available wipes are not considered nontoxic food additives grade or GRAS. The carrier fabric of the wipes also can have absorptive and abrasive properties to remove soil and clean the surface. However, the fabric material may not be biodegradable and recyclable. Biodegradability and recycling properties are crucial for planning a prolonged space journey to in the outer space.

The sanitizing wipes of this invention are free of all the above undesirable properties. The sanitizing composition hereof incorporates enhanced microbicidal properties of either (a) anionic surface-active agents or (b) that a mixture of organic acids at acidic pH below neutrality. The FDA in 21 CFR lists these ingredients as either food additive grade or GRAS. The fabric material of these wipes is based on cellulose or other natural biodegradable polymers.

The present invention also eliminates the use and attendant cost of additional antimicrobial agents while delivering sanitizing and microbicidal properties.

### SUMMARY OF THE INVENTION

Generally, the present invention comprises a wipe having the microbicidal or germ killing properties of food additive anionic surface-active agents and those of compounds that are Generally Regarded As Safe (GRAS) at pH values below 7.0. The anionic surface-active agents used herein serve both as cleaning agents as well as microbicidal agents. For use in wipes the anionic surface-active agent must be present in optimum amount for cleaning purposes.

The present invention provides a safe sanitizing and microbicidal wipe without the use of compounds that are toxic, corrosive or which leave substantial residue on the surface. Further, the present invention is disinfecting and sanitizing wipe composition that is economical, useful for preventing infections in everyday life without the drawbacks of antibacterial as well as sanitizing wipe products. The wipe hereof generally comprises:
(a) a mixture of (1) at least one food additive grade or GRAS anionic surface-active agent and (2) at least one food grade acid to lower the pH at or below 7.0, and
(b) a biodegradable synthetic or natural material carrier fabric, the mixture being deposited on or impregnated in the fabric.

The mixture may further comprise:
(3) a food grade sequestering agent and,
(4) solubilizing agent.

In accordance herewith, there is provided a class of chemical agents that can be used to prepare the antimicrobial, sanitizing and/or disinfecting wipes for cleaning, and sanitizing or disinfecting environmental

The anionic surface-active agent used herein has the following general formula:
R-An−Cn+
wherein R is a hydrophobic group selected from the group consisting of substituted or unsubstituted n-alkyl, n-alkenyl, alkylbenzyl, or alkylnapthalene group with length equivalent to 6 to 16 carbon atoms; A is an anionic group selected from the group consisting of monocarboxylic, dicarboxylic, sulfate, sulfonate, phosphate and phosphonate or mixtures thereof; C is a cationic group capable of forming an electrostatic linkage with A, and n represents the number of ionic charges.

The anionic agent may be used in concentrations ranging from about 10 ppm to about up to 100,000 ppm. The anionic agent can be used as a single molecular species or in combination with other anionic molecular species.

Other foam generating agents and surface tension reducing agents that help the cleaning process may be incorporated in the composition without affecting the germ killing property.

The food grade acid may include, for example, adipic acid, ascorbic acid, benzoic acid, citric acid, dehydroacetic acid, erythorbic acid, fumaric acid, glutaric acid, gluconic acid, hyaluronic acid, hydroxyacetic acid, lactic acid, malic acid, salicylic acid, sorbic acid, succinic acid, adipic acid, pimelic acid, suberic acid, azelaic acid, sebacic acid, tannic acid, tartaric acid, phosphoric acid, carboxylic acid polymers, homo- or hetero-polymerized carboxylic acid such as polylactic acid or polylactic-glycollic acid; and the like, as well as mixtures thereof.

The sequestering agent can be citric acid, sodium gluconate, isopropyl citrate and EDTA, sodium acid phosphate, sodium acid pyrophosphate or other suitable food grade-sequestering agent, as well as mixtures thereof.

Suitable solubilizing agents compatible with the carrier fabric of the wipes include water and/or various alcohols such as ethyl, propyl, isopropyl alcohols or glycols or mixtures thereof.

The biodegradable carrier wipe fabric material is prepared from natural and/or synthetic biodegradable and recyclable polymers including cellulose and modified cellulose.

For a more complete understanding of the present invention reference is made to the following detailed description and accompanying examples:

### DESCRIPTION OF THE PREFERRED EMBODIMENT

As hereinabove noted the present invention comprises a sanitizing and microbicidal wipe. The wipe hereof generally comprises:
(a) a mixture of (1) at least one food additive grade or GRAS anionic surface-active agent and (2) at least one food grade acid to lower the pH at or below 7.0, and
(b) a biodegradable synthetic or natural material carrier fabric, the mixture being deposited on or impregnated in the fabric.

The mixture may further comprise:
(3) a food grade sequestering agent and,
(4) solubilizing agent.

More particularly, the anionic surface-active agent contemplated for use herein is either the acid or salt form of the following classes of compounds:
(a) C₆-C₁₈ alkyl- and alkenyl-sulfates;
(b) C₆-C₁₈ alkyl- and alkenyl-ether sulfates;
(c) C₈-C₁₆ alkyl diphenyl ether disulfonates;
(d) C₄-C₁₈ fatty acid isethionates;
(e) C₈-C₁₆ alkyl- and alkenyl sulfonates;
(f) dialkyl- and dialkenyl sulfosuccinates in which the alkyl or alkenyl groups independently contain from six to eighteen carbon atoms;
(g) C₆-C₁₈ alkylbenzene sulfonates;
(h) naphthalene sulfonates;
(i) alkyl naphthalene sulfonates in which alkyl group contains from one to six carbon atoms;
(j) the mono-(n-alkyl) and mono-(n-alkenyl) acyl esters of C₂-C₄ hydroxylated monocarboxylic acids in which the alkyl or alkenyl group contains from six to eighteen carbon atoms;
(k) the mono-(n-alkyl) and mono-(n-alkenyl) acyl esters of C₂-C₄ hydroxylated dicarboxylic acids in which the alkyl or alkenyl group contains from six to eighteen carbon atoms;
(l) the mono-(n-alkyl) and mono-(n-alkenyl) alkyl esters of C₂-C₄ dicarboxylic acids, in which the alkyl or alkenyl group contains from six to eighteen carbon atoms; and
(m) C₄-C₁₈ fatty alcohol sulfoacetates.

The anionic agent used in the sanitizing and microbicidal wipe composition is, preferably, either sodium dodecyl sulfate, sodium dioctyl sulfosuccinate, sodium decyl lactylate, sodium dodecylbenzene sulfonate, and sodium alpha-olefin sulfonate and mixtures thereof.

Generally, the concentration of anionic agent/agents is from about 0.0010% w/v to about 20.0% w/v.

The acidifying agent is used to lower the pH below 7.0, and, preferably, at or around pH 3.0. The acidifying agent used to lower the pH must be compatible with the anionic surface-active agent. The acidifying agent is, therefore, one or more of adipic acid, ascorbic acid, citric acid, dehydroacetic acid, erythorbic acid, fumaric acid, glutaric acid, gluconic acid, hyaluronic acid, hydroxyacetic acid, lactic acid, malic acid, succinic acid, tannic acid, tartaric acid, adipic acid, pimelic acid, suberic acid. azelaic acid. sebacic acid, carboxylic acid polymers, homo- or hetero-polymerized carboxylic acid such as poly lactic acid or poly lactic-glycolic acid, as well as mixtures thereof.

The preferred acidifying agent is either citric acid, benzoic acid, sorbic acid, adipic acid, or lactic acid or mixtures thereof. The acidifying agent is present in at concentrations from about 0.01% w/v to about 10% w/v.

As noted the composition has one or more sequestering agents included therewith. Representative sequestrants are, for example, citric acid, sodium gluconate, isopropyl citrate, EDTA, sodium acid phosphate, sodium acid pyrophosphate and the like food grade sequestering agents, as well as mixtures thereof.

Citric acid is a dual action component serving as an acidifying agent and a sequestering agent.

Additional food grade suitable sequestering agents can, also be incorporated. The preferred sequestering agent is either citric acid, sodium EDTA or sodium acid pyrophosphate and mixtures thereof.

The solubilizing agent can be selected from the group consisting of water and/or various alkanols and glycols such as ethyl, propyl, isopropyl alcohols or glycols.

Where used, the alcohol can serve as both a solubilizing agent as well as a quick drying agent. However, the preferred solubilizer used is deionized or distilled water to the solubilizer is used in an amount 100% w/v of the composition.

The biodegradable carrier wipe fabric material is preferably a biodegradable natural and/or synthetic material such as cellulose and modified cellulose. The preferred fabric material is cellulose-based.

Other foam generating agents and surface tension reducing agents that help the cleaning process may be incorporated in the composition without affecting the germ killing property.

The composition may include other compatible ingredients, which do not reduce or interfere with the antimicrobial and cleaning properties. The composition may additionally include an emollient such as aloe vera, a coloring agent, polymeric antioxidants, fragrances, vitamins, and moisturizing agents for importing a soft and silky feel to the skin.

The composition may, also, optionally contain sodium chloride at levels of from about 0.5% w/v to about 5% w/v.

The preferred skin-conditioning agent used is the powdered extract of aloe vera used at concentrations of between about 0.05% w/v to about 2% w/v.

The sanitizing wipes can be fortified with antimicrobial agents such as benzoic acid, sorbic acid, alkyl lactates, such as, methyl lactate, ethyl lactate, propyl lactate, butyl lactate and the like, alkyl lactylates, such as, octyl lactylate, decyl lactylate, lauryl lactylate and the like, which may be used independently at concentrations of from about 0.01% w/v to about 5.0% w/v.

To be more useful in an environment where mycobacteria might be present, the sanitizing wipes composition can also be prepared with tuberculocidal activity such as by using decyl lactylate. This example illustrates the efficacy of the present invention in a mycobacterial.

The antimicrobial composition can be prepared either as concentrated form to be diluted for depositing onto the wipes by suitable dilution or as ready-to-use solution for directly depositing onto the wipes. Deposition methods can be spraying, dipping, passing the fabric through a solution as required to wet the wipes. The concentrate form can also be deposited on the carrier fabric when making the fabric. In this case the dry wipes become microbicidal wipes when activated with water or other solubilizing agent. These wipes can be available in a roll stock as bathroom tissue.

For a more complete understanding of the present invention reference is made to the following illustrative, non-limitative examples. In the examples all percentages are by w/v, absent contrary indications. The following examples show that the sanitizing compositions used for depositing on the wipes have effective microbicidal properties for using the wipes as sanitizing or disinfecting wipes.

### EXAMPLES I-III

To ascertain the sanitizing efficacy and microbicidal spectrum against bacteria, three sanitizer compositions were first evaluated by the sanitizer and germicidal detergent test specified by the Association of Analytical Chemists (A.O.A.C.), 1984, using Escherichia coli ATCC 11229 and Staphylococcus aureus ATCC 6538. The bacterial cultures were purchased from the American Type Culture Collection (ATCC), (Bethesda, Md.). Escherichia coli and Staphylococcus aureus are used to represent both gram negative and gram-positive bacterial classes. This test is used by the US EPA for to determine compliance for a product to be used as a sanitizing agent. A product that reduces population density of both types of bacteria in 30 s by 99.999% satisfies the requirement as a sanitizing agent. Fifty ppm hypochlorite meets the criterion of a sanitizer in this test against Escherichia coli and Staphylococcus aureus and was used as a positive control. The results are shown in Table I.

The efficacy of the sanitizers of Examples I-III were further evaluated with 50 ppm of hypochlorite as a control against Salmonella typhimurium, Listeria monocytogenes, Aeromonas hydrophila, and Pseudomonas aeruginosa essentially by the modified detergent and germicidal sanitizer test of the A.O.A.C. (Lopes, 1986) as shown in Table 2. The sanitizers were tested against both natural isolates and the antibiotic resistant strains of Listeria monocytogenes and Salmonella typhimurium. Each of the antibiotic resistant strains was used separately as well as in a composite strain prepared by mixing equal numbers of five different antibiotic resistant strains of Listeria monocytogenes or those of Salmonella typhimurium, respectively.

TABLE 1

| Sanitizing | | | % Kill | |
|---|---|---|---|---|
| Composition | Ingredient | % w/v | Staph. aureus | E. coli |
| Example 1 | Citric acid | 0.37 | >99.999 | >99.999 |
| | Sodium dodecyl | 0.028 | | |
| | sulfate | | | |
| Example 2 | Acetic acid | 0.97 | >99.999 | >99.999 |
| | Dioctyl Sulfosuccinate | 0.029 | | |
| Example 3 | Lactic Acid | 0.85 | >99.999 | >99.999 |
| | Decyl Lactylate | 0.029 | | |
| Control | Hypochlorite | 50 ppm | >99.999 | >99.999 |

TABLE 2

| Test | | Number of surviving bacteria/ml after | | | |
|---|---|---|---|---|---|
| organism & | Test | 30 seconds | | 60 seconds | |
| ATCC # | Sanitizer | cfu/ml | % Kill | cfu/ml | % Kill |
| A.h. 7965 | Example 1 | 0, 0 | >99.999 | 0, 0 | >99.999 |
| L.m. 7644 | | 2, 3 | >99.999 | 0, 0 | >99.999 |
| P.a. 10145 | | 0, 0 | >99.999 | 0, 0 | >99.999 |
| S.t. 7823 | | 0, 0 | >99.999 | 0, 0 | >99.999 |
| A.h. 7965 | Example 2 | 4, 2 | >99.999 | 0, 0 | >99.999 |
| L.m. 7644 | | 0, 0 | >99.999 | 0, 0 | >99.999 |
| P.a. 10145 | | 0, 0 | >99.999 | 0, 0 | >99.999 |
| S.t. 7823 | | 1, 0 | >99.999 | 0, 0 | >99.999 |
| A.h. 7965 | Example 3 | 0, 0 | >99.999 | 0, 0 | >99.999 |
| L.m. 7644 | | 0, 0 | >99.999 | 0, 0 | >99.999 |
| P.a. 10145 | | 7, 1 | >99.999 | 1, 2 | >99.999 |
| S.t. 7823 | | | >99.999 | 0, 0 | >99.999 |
| A.h. 7965 | Hypochlorite | 1*, 0* | >99.999 | 0, 0 | >99.999 |
| L.m. 7644 | | 82*, 60* | >99.999 | 17, 16 | >99.999 |
| P.a. 10145 | | 1, 2 | >99.999 | 1, 0 | >99.999 |
| S.t. 7823 | | T@, T@ | <99.999 | 86, 94 | >99.999 |

*HOCL = 200 ppm.
@HOCL = 50 ppm.
T = Too Numerous To Count.
A.h. = Aeromonas hydrophila,
L.m. = Listeria monocytogenes,
P.a. = Pseudomonas aeruginosa,
S.t. = Salmonella typhimurium.

### EXAMPLE IV

The composition of Example 3 was tested tuberculocidal activity. An aqueous solution of 300 ppm of decyl lactylate and 8800 ppm of lactic acid (pH≦3.0) exhibited higher tuberculocidal activity than 100 ppm of hypochlorite when examined essentially by the AOAC germicidal and detergent sanitizer test (Table 2). The tests were carried out in 500 ppm of hard water prepared according to the AOAC procedure (AOAC, 1990). The neutralizing solution used was a solution of lecithin with polysorbate 80 prepared in phosphate buffer pH 7.2. In case of hypochlorite 0.1 ml of 10% thiosulfate was added to each tube of the neutralizer solution.

The culture was prepared according to the AOAC procedure for testing tuberculocidal activity of disinfectants (AOAC, 1990). Mycobacterium tuberculosis H37 Ra was maintained on Middlebrook 7H9 agar consisting of 4.7 g Middlebrook 7H9 broth (Difco), 2 ml of glycerol (Sigma) and 15 g Bacto-Agar in 900 ml of deionized water and 100 ml of Middlebrook ADC enrichment (Difco). The same medium was used for surface plating during the test. To provide aerobic conditions for Mycobacterium tuberculosis surface plating was used on pre-poured plates. Pre-poured plates were dried by storing at room temperature for one week prior to use.

The culture from a 20-day-old slant was inoculated into a modified Proskauer-Beck broth and incubated undisturbed at 37 C for 25 days. The culture from a single tube was homogenized to a smooth suspension free of visible clumps with 1 ml of 0.1% Tween 80 in 0.9% NaCl. The culture was diluted to give 20% transmission at 650 nm and used in the test.

Germicidal and detergent sanitizer test (AOAC, 1990) was essentially employed for evaluating tuberculocidal of decyl lactylate. Ninety-nine ml of the test sanitizer solution contained 300 ppm of decyl lactylate and 8800 ppm of lactic acid. The test was carried out by rapidly mixing 1 ml of the test suspension into 99 ml of the sanitizer solution. After contact period of 30″, 60″, and 5′, one ml of the test mixture was removed and added to 9 ml of the neutralizer. Clorox at 100 ppm was used as positive control. One ml of the neutralized test mixture was plated on Middlebrook 7H9 agar. The culture was diluted ten fold with 0.02% Tween 80 and plated to estimate the number of bacteria used in the test. The plates were replaced in the original plastic plate bags, which were sealed with adhesive tape to prevent drying and incubated at 37 C for three weeks. The colonies were counted and reduction in colony count compared to controls was used to calculate efficacy of the sanitizers.

The tests show microbicidal efficacy of sanitizing wipe compositions against gram positive, gram negative as well as tuberculosis bacteria. The tests show that the sanitizing composition has a broad-spectrum lethal activity against microorganisms. Thus the lethal activity of the sanitizing wipes can be used to disinfect surfaces contaminated with bacteria, fungi, protozoa and viruses.

The sanitizing compositions are deposited on biodegradable fabric obtained from Ahlstrom Windosr Locks, LLC. The biodegradable Grade 10180 and Grade 10201 fabrics were used for the preparation of sanitizing wipes for vegetables and environmental surfaces. Grade 10234 fabric was used for personal care sanitizing wipes. Thus the sanitizing compositions can be used to prepare biodegradable sanitizing wipes for environmental and food contact surfaces as well as for personal care use.

TABLE 3

| | CFU After Contact Time | | |
|---|---|---|---|
| | in Seconds/minutes | | |
| | 30 Seconds | 60 Seconds | 5 Minutes |
| Example 3 | % Kill | % Kill | % Kill |
| Decyl lactylate (0.029%) | ≧99.996 | ≧99.999 | ≧99.999 |
| | ≧99.996 | ≧99.999 | ≧99.999 |
| NaOCl (100 ppm) | ND | ND | ≧99.998 |
| | ND | ND | ≧99.997 |

Activity of decyl lactylate under acidic pH (<3.0);
CFU = colony forming unit,
ND = Not determined,
T = Too Numerous To Count (TNTC),
Challenge Number of CFUs/ml = 72 × 10⁶

Having thus described the invention, what is claimed is:

1. A sanitizing and microbicidal wipe for cleaning and disinfecting surface comprising: (a) a mixture of (1) food additive and/or generally regarded as safe anionic surface-active agent and (2) a food grade acidifying agent and (b) a carrier fabric, the mixture being deposited on the fabric.

2. The wipe of claim 1 wherein the final pH of the mixture is below pH 7.0.

3. The wipe of claim 1, wherein the acidifying agent is selected from the group consisting of adipic acid, ascorbic acid, citric acid, dehydroacetic acid, erythorbic acid, fumaric acid, glutaric acid, gluconic acid, hyaluronic acid, hydroxyacetic acid, lactic acid, malic acid, succinic acid, tannic acid, tartaric acid, adipic acid, pimelic acid, suberic acid. azelaic acid. sebacic acid, carboxylic acid polymers, homo- or hetero-polymerized carboxylic acid and mixtures thereof.

4. The wipe of claim 3 wherein the acidifying agent is selected from the group consisting of citric acid, lactic acid, ascorbic acid, adipic acid and mixtures thereof.

5. The wipe of claim 1 wherein the anionic surface-active agent is selected from the group consisting of:
a. C₆-C₁₈ alkyl- and alkenyl-sulfates;
b. C₆-C₁₈ alkyl- and alkenyl-ether sulfates,
c. C₈C₁₆alkyl diphenyl ether disulfonates,
d. C₄C₁₈ fatty acid isethionates;
e. C₆-C₁₈ alkyl- and alkenyl sulfonates;
f. dialkyl- and dialkenyl sulfosuccinates in which the alkyl or alkenyl groups independently contain from six to eighteen carbon atoms;
g. C₆-C₁₈ alkylbenzene sulfonates;
h. naphthalene sulfonates;
i. alkyl naphthalene sulfonates in which alkyl group contains from one to six carbons atoms;
j. the mono-(n-alkyl) and mono-(n-alkenyl) acyl esters of C₂-C₄ hydroxylated monocarboxylic acids in which the alkyl or alkenyl group contains from six to eighteen carbon atoms;
k. the mono-(n-alkyl) and mono-(n-alkenyl) acyl esters of C₂-C₄ hydroxylated dicarboxylic acids in which the alkyl or alkenyl group contains six to eighteen carbon atoms;
l. the mono-(n-alkyl) and mono-(n-alkenyl) alkyl esters of C₂-C₄ dicarboxylic acids, in which the alkyl or alkenyl group contains from six to eighteen carbon atoms; and
m. C₄-C₁₈ fatty alcohol sulfoacetates, the anionic surfactant being present as either the free acid or salt thereof.

5. The wipe of claim 4 wherein the anionic surface-active agent is concentration range from about 0.001% w/v to about 20% w/v.

6. The wipe of claim 4 wherein the anionic surfactant is a food additive ingredient.

7. The wipe of claim 4 wherein the anionic surfactant is selected from the group consisting of sodium dodecyl sulfate, sodium decyl lactylate, and sodium dioctyl sulfosuccinate and mixtures thereof.

8. The wipe of claim 1 wherein the mixture further comprises: a sequestering agent, the sequestering agent being selected from the group consisting of citric acid, sodium gluconate, isopropyl citrate, EDTA, sodium acid phosphate, sodium acid pyrophosphate and mixtures thereof.

9. The wipe of claim 8 wherein the sequestering agent is selected from the group consisting of citric acid, sodium gluconate, sodium acid pyrophosphate, EDTA and mixtures thereof.

10. The wipe of claim 9 wherein the sequestering agent is present in a concentration of from about 0.001% to about 10% w/v.

11. The wipe of claim 1 which further comprises a solubilizing agent selected from the group consisting of water, ethyl alcohol, propylene glycol and mixtures thereof.

12. The wipe of claim 1 wherein the carrier fabric is a biodegradable natural or synthetic material or a mixture thereof.

13. The wipe of claim 1 which further comprises: a skin conditioning agent, the skin conditioning agent being selected from the group consisting of Vitamin A, Vitamin C, Vitamin D and Vitamin E; menthol, thymol, tea tree oil, natural plant extract, sea weed extracts, protein hydrolysates, fatty alcohols, fatty alcohol esters, sphingolipids and mixtures thereof, the skin conditioning agent being deposited on the fabric.

14. The wipe of claim 13 wherein the skin conditioning agent is an extract of Aloe vera plant.

15. The wipe of claim 14 wherein the skin conditioning agent is present at a concentration of between about 0.01% w/v to about 5.0% w/v.
